(19) United States
(12) Patent Application Publication
(10) Pub. No.: US 2024/0325577 A1
(43) Pub. Date: Oct. 3, 2024

(54) SANITATION DEVICE FOR STETHOSCOPE

(71) Applicant: ORLANDO HEALTH, INC., Orlando, FL (US)

(72) Inventors: Mitch Barneck, Orlando, FL (US); Timothy B. Bullard, Orlando, FL (US); Michael J. Schmidt, Maitland, FL (US); Ran Zhou, Lake Nona, FL (US); Fred Maxik, Lake Nona, FL (US); Michael Miller, Lake Nona, FL (US)

(73) Assignee: ORLANDO HEALTH, INC., Orlando, FL (US)

(21) Appl. No.: 18/625,663

(22) Filed: Apr. 3, 2024

Related U.S. Application Data
(60) Provisional application No. 63/493,858, filed on Apr. 3, 2023.

(51) Int. Cl. A61L 2/10; A61B 7/02

(52) U.S. Cl. CPC A61L 2/10; A61B 7/02; A61L 2202/121; A61L 2202/24

(57) ABSTRACT

A stethoscope sanitizing device for a stethoscope may have a housing that at least partially surrounds a sanitation cavity. The stethoscope sanitizing device also may include a radiation emitter located within the housing, where the radiation emitter is configured to selectively emit a radiation into the sanitation cavity. The device also may include a support pane fixed to the housing and configured to receive and support a patient-facing side of a stethoscope.

### CROSS-REFERENCE TO RELATED APPLICATION

This application claims priority to U.S. Provisional Application No. 63/493,858, filed on Apr. 3, 2023, the entirety of which is hereby fully incorporated by reference herein.

### TECHNICAL FIELD

The present disclosure relates to a sanitation device for a stethoscope, particularly a device for sanitizing a patient-facing surface of the stethoscope via a radiation emitter.

### BACKGROUND

Stethoscopes are among the most commonly used medical instruments in a healthcare setting. Stethoscopes function by detecting sounds produced by a patient's organs and relaying such sounds to a medical professional. In view of their extensive use (and since a single stethoscope may be used on multiple patients), stethoscopes may cross-infect patients when they are not properly cleaned (e.g., sanitized or sterilized). Even in view of this issue, it is rare for physicians to sanitize his or her stethoscope between patients, particularly since few portable, quick, and easy-to-use sanitation devices exist in the prior art.

### BRIEF DESCRIPTION OF THE DRAWINGS

The embodiments discussed herein may be better understood with reference to the following drawings and description. Certain features shown in the figures are not necessarily to scale. Moreover, in the figures, like-referenced numerals designate corresponding parts throughout the different views.

FIG. 1 is an illustration showing a perspective view of a stethoscope sanitation device attached to a stethoscope in accordance with certain aspects of the present disclosure.

FIG. 2 is an illustration showing a perspective view of the stethoscope sanitation device from FIG. 1.

FIG. 3 is an illustration showing an exploded view of the stethoscope sanitation device from FIG. 1.

FIGS. 4-7 are illustrations showing various views (front, side, back, and bottom) of the stethoscope sanitation device from FIG. 1.

### DETAILED DESCRIPTION

In view of the background above, the present embodiments relate to a stethoscope sanitation device that is portable, quick, easy to use, and cost effective. Such devices have the potential to be widely-used in healthcare settings, which may decrease cross-infections between patients.

In this document, “sanitize” and “sanitation” means reducing or preventing microorganisms on a surface (e.g., a stethoscope surface) to ensure the number and density of microorganisms is at a safe level in an attempt to prevent cross-infections between patients. “Sterilization” is included within the definition of sanitation (where sterilization is narrower in meaning, particularly in that sterilization includes removing substantially all of the living microorganisms on a surface). Also, in this document, “cleaning” generally means “sanitizing.”

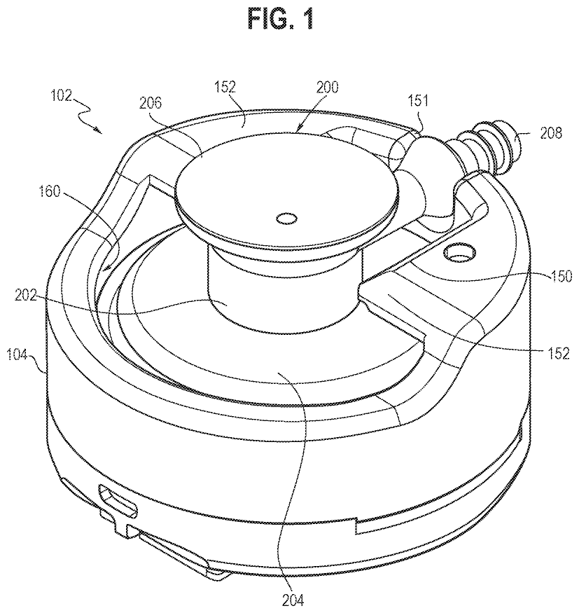

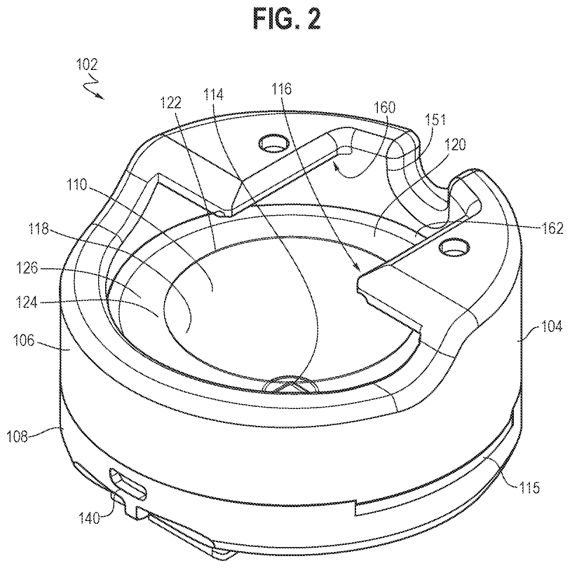

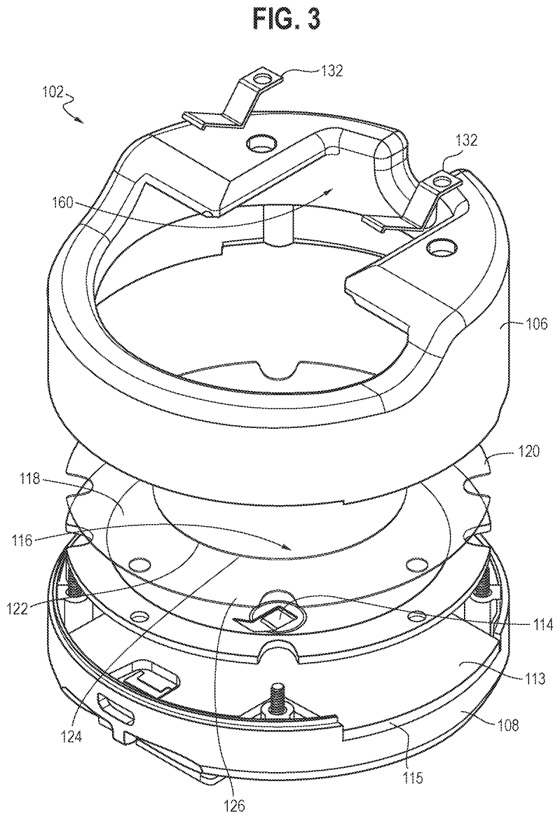

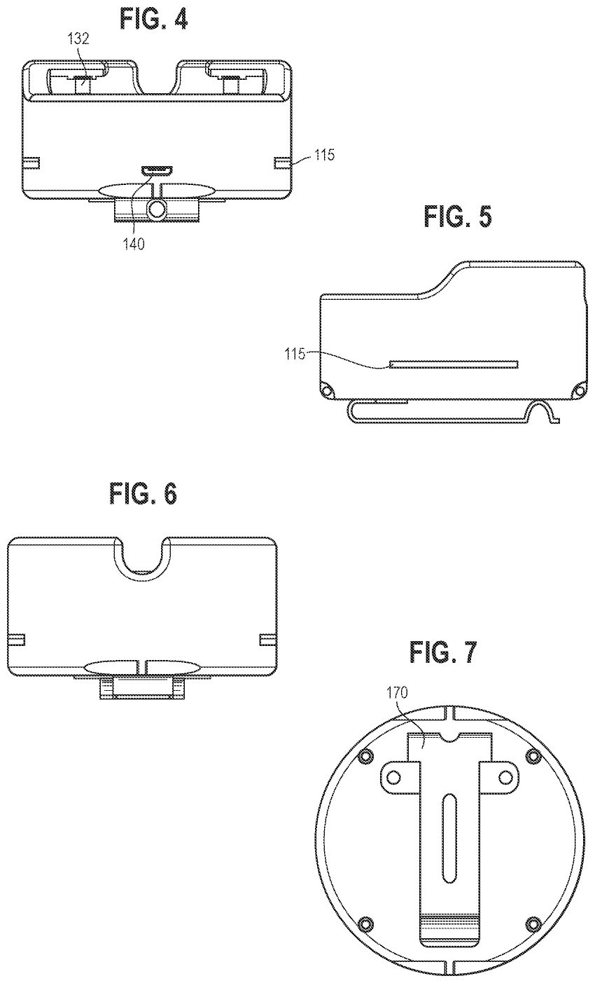

FIGS. 1-7 show an example of a stethoscope sanitation device 102. The stethoscope sanitation device 102 is generally formed by a housing 104, which includes a top piece 106 and a bottom piece 108. Notably, “top” and “bottom” are used only for convenience and simplicity of description in this document (including in the claims), and “top” as used herein is defined as the side of the device that engages a stethoscope 200. Thus, the top piece 106 of the housing 104 has certain features for receiving and securing to the stethoscope 200, and particular a chest piece 202 of the stethoscope 200, as discussed in more detail below. The bottom piece 108 of the housing 104 may generally cover a controller, light guide 113, battery, UV-emission equipment, and/or any other suitable objects located within the device which are intended to remain generally inaccessible to the end user.

A sanitation cavity 110 may be formed at least partially within the housing 104, which may be a cavity where sanitation of the stethoscope generally takes place. For example, a radiation emitter 114 may be positioned within the housing 104 such that, when turned on (i.e., in an emitting state), the radiation emitter 114 may selectively emit radiation waves (such as UV light waves) into the sanitation cavity 110. These radiation waves may be substantially directed towards a diaphragm 204 of the stethoscope 200 in an effort to kill or disable pathogens on the patient-facing surface of the diaphragm 204 and other surfaces of the stethoscope 200. In certain exemplary embodiments, the radiation emitter 114 may be (or include) a solid-state UV source, which may have high reliability, may have low maintenance needs, may lack toxins or heavy metals, and may include other advantages relative to other radiation sources. Other radiation emitters may be alternatively or additionally included.

A reflective surface 116 may generally surround the radiation emitter 114, as shown in the figures. The reflective surface 116 is optional, as the radiation emitter 114 may be capable of providing radiation to the entire desired surface without reflections. However, when included, the reflective surface 116 may form a majority of, and substantially all of, the surfaces that will be exposed within the sanitation cavity 110 when the stethoscope 200 is in a sanitation position. The reflective surface 116 may generally direct radiation waves towards the stethoscope as they are emitted within the sanitation cavity.

The reflective surface 116, when included, may form an inverted cone 118, which may generally form the bottom of the sanitation cavity 110. The inverted cone 118 may be included even if not reflective, although the inventors contemplate embodiments where the inverted cone is not needed at all. However, when included, the inverted cone may be advantageous for direct reflected radiation rays within the sanitation cavity 110 towards the diaphragm of the stethoscope, and may be shaped such that the interior walls of the inverted cone do not block direct radiation from the emitter as it moves towards the stethoscope. Optionally, the radiation emitter 114 may be located at an apex of the inverted cone 118 such that the radiation emitter is substantially centered relative to the inverted cone 118, thereby facilitating a uniform distribution of radiation across the patient-facing side of the stethoscope 200.

The device 102 may include a support pane 120 fixed to the housing and configured to receive and support a patient-facing side of a stethoscope 200. Advantageously, the support pane 120 may act as a barrier between the stethoscope 200 and the area inside the inverted cone 118, thereby preventing the stethoscope 200 from entering the inverted cone 118 (where it would be difficult to position in a stable manner). Preferably (but not necessarily), the support pane 120 is a substantially flat sheet of transparent material that is sized, shaped, and oriented to at least partially cover the interior of the inverted cone within the sanitation cavity 110. Without limitation, the support pane 120 may be formed of an FEP material (i.e., fluorinated ethylene propylene), which is particularly suitable since FEP includes a low UV resistance and high UV transmittance, meaning UV waves easily pass through the FEP material relative to other transparent material types. Thus, radiation emitted within the sanitation cavity may easily penetrate the support pane 120 as they move towards the stethoscope, and the support pane 120 may not present a substantial obstacle blocking such radiation. In certain non-limiting embodiments, at least 75% of radiation from the emitter directed towards the support pane 120 passes through and reaches the stethoscope, for example, such as at least 80%, and even more than 90%.

The support pane 120 may be flat, but in other embodiments it may be angled/sloped, curved, or otherwise have non-flat characteristics for a particular function. For example, the support pane 120 may be slightly convex in shape from the top perspective (meaning the center-most portions of the support pane 120 are closer to the bottom of the device than the outer-most portions of the support pane 120). Advantageously, this curvature may facilitate centering of a stethoscope placed on the support pane 120, as the stethoscope 200 will have a tendency to move towards a centralized position when pressed against the support pane 120. In other embodiments (perhaps in addition to the curved support pane 120), the stethoscope sanitation device 102 may have other features for centering the stethoscope, such as abutment surfaces, springs, protrusions, or the like.

In some embodiments, including the depicted embodiment, the support pane 120 includes a circular (or other shaped) opening 122, which may be substantially centered relative to the radiation emitter 114 and/or the central axis of the inverted cone 118. The opening 122 may expose a substantial portion of the stethoscope's diaphragm to direct radiation emitted by the radiation emitter 114 without passing through the transparent material, for example, which may further enhance the sanitation performance of the device (even when the transparent material is highly transmissible). In other words, this opening 122 may be advantageous for limiting the UV-filtering effect of the support pane 120. In other embodiments (which are not depicted in the figures), the support pane 120 may lack a primary opening such that it substantially covers the interior of the inverted cone 118 from the perspective of the stethoscope's diaphragm.

When the opening 122 of the support pane 120 is included, an inner perimeter area 124 that is adjacent to, and surrounding, the opening 122 may be configured (e.g., sized, shaped, positioned, and constructed) for direct contact with the patient-facing side of the stethoscope 200. In certain exemplary embodiments, the diameter of the opening is small enough, such as smaller than the diameter of a relatively small stethoscope diaphragm, that the inner perimeter area 124 of the support pane 120 may block a relatively small stethoscope from falling into the inverted cone (which may compromise sanitation).

By contrast, an outer perimeter area 126 may be radially outside the inner perimeter area 124, and may have a diameter that is larger than relatively large size(s) of commonly-used stethoscopes. Advantageously, this feature provides the ability for one stethoscope cleaning device to be applicable to many different stethoscope sizes (and types). This may make the stethoscope sanitation device 102 more marketable and lower its cost overall in view of manufacturing scaling. The inner perimeter area 124 and the outer perimeter area 126 may be collectively referred to as a “support ring” herein.

Notably, while the opening 122, the inner perimeter area 124, and the outer perimeter area 126 are depicted as having circular perimeter dimensions, this is not necessary. Other shapes are also contemplated, whether to accommodate stethoscopes having a particular shape, for enhanced portability, for manufacturing and/or assembly convenience, for aesthetic reasons, or the like.

To secure the stethoscope 200 in place (e.g., with its patient-facing side abutting the support pane 120), the device 102 may include certain holding or support features configured for gripping the chest piece 202 of the stethoscope 200. For example, the top piece 106 of the housing 104 may include a slot 150 for accommodating a central portion of the chest piece 202 that extends between the stethoscope's diaphragm 204 and bell 206, along with a stem slot 151 that receives the tubing 208 extending from the stethoscope's chest piece 202. Other top portions 152 of the top piece 106 may be generally located between the stethoscope's diaphragm and bell when the stethoscope is positioned for sanitation.

The structures discussed in the paragraph above may be sufficient alone to hold the stethoscope 200 in its proper position in certain circumstances, such as (but not limited to) when the device 102 is hung on a vertical surface with the “top” of the device facing horizontally (meaning gravity will retain the stethoscope's receipt within the slot 150). In other embodiments, the top piece 106 of the housing may have a flexible or compliant material that is displaced when the stethoscope 200 is inserted, for example. It is also contemplated that magnets, spring legs, or other features may be included to retain suitable positioning.

In the depicted example, the device 102 includes two spring legs 132 that extend from the inner-facing surface of the top piece 106 of the housing 104. These spring legs 132 may have one end fixed to the housing and one free end, where the free end is displaceable upon receiving a force such that the stethoscope 200 can slide between the free end of the spring legs 132 and the housing 104. While two spring legs 132 are included, more or fewer may be included in other embodiments.

When the stethoscope 200 is secured by the device 102, a rim 160 of the device may substantially prevent radiation from escaping the sanitation cavity 110. The rim 160 may extend axially beyond the top of the support pane 120, as shown, meaning that the rim 160 extends axially beyond the patient-facing diaphragm surface when the stethoscope 200 is fixed in place. Thus, when a relatively small stethoscope is used that does not cover the entirety of the support pane 120, all or most radiation waves that would otherwise escape from the device (e.g., due to a gap between the outer-most perimeter of the support pane 120 and an outer-most dimension of the stethoscope 200) are blocked from escaping by the raised structure provided by the rim 160. As shown, the rim 160 is circular in shape and extends around the entirety of the device 102, although this is not required in all embodiments.

Optionally, a non-transparent lower rim 162 may be included adjacent to the rim 160, which may be substantially flush with the support pane 120. The lower rim 162 may create radial space between the above-mentioned potential gap where radiation may escape and the rim 160. The lower rim 162 may be immediately adjacent to the inverted cone of the device.

To enhance the portability of the device 102, the housing 104 may encompass several other features beneath the inverted cone. For example, a battery holder, a circuit board (not shown) forming the controller, radiation emission equipment, and any other suitable feature may be located within the housing 104, perhaps fixed to the bottom piece 108 of the housing 104. The bottom piece of the housing 104 may be removeable from the top piece 106 to access these devices, which may allow for battery replacement, bulb replacement, or other repairs or replacements as needed. Optionally, a belt engagement structure 170 or other structure may be included for securing the device at an easily accessible location within a medical room, or on a medical professional's clothing, body, or equipment may be included to increase the use of the device via ensuring it is immediately available when needed.

The bottom piece 108 of the housing may also include a connection port 140 for receiving a wired connection, which may be used to charge the battery, send or receive data (e.g., to adjust the sanitation methodology via changing the timing, frequency, or other characteristics of the radiation emitter).

In some embodiments, a light guide 113 may be included. The light guide may be formed of a material that glows upon receipt of UV rays, such as a triphosphoric material or another suitable material. Thus, if at least a portion of the light guide 113 is positioned to absorb radiation from the radiation emitter 114, the light guide 113 may glow. The light guide 113 may further include one or more indication tabs 115 that are exposed to view such that, when the device is emitting radiation, the indication tabs 115 of the light guide 113 indicates to a user that the device is operating. This embodiment may be advantageous for providing a direct indication of the device's operation without relying on sensors, control algorithms within a controller connected to indicator lights, or the like.

A controller of the device may be included and electrically connected to the radiation emitter 114 to control its operation. For example, the controller may be connected to either a sensor for detecting the stethoscope's position, and “on/off” button, or both. The controller may also generally control the length and intensity of radiation emission, the frequency of sanitation procedures (e.g., where such procedures are repeated periodically), or the like. In certain embodiments, an on/off button may be mechanically coupled to the housing 104 such that it is activated, perhaps via stethoscope abutment, when the stethoscope is placed in an appropriate position for sanitation. This button may activate certain sanitation processes at the will of the controller. While not shown, the device may include various safety switches, buttons, or other interfaces where an operator can intervene if the device is not working properly. As will be appreciated by those skilled in the art, there are limitless control aspects that may be implemented within the controller with regards to the operation fo the emitter and/or other features of the device. As mentioned above, the controller may be connected via the connection port 140 such that certain parameters can be adjusted or implemented via computer connection, although this is optional.

While various embodiments have been described, it will be apparent to those of ordinary skill in the art that many more embodiments and implementations are possible. Accordingly, the embodiments described herein are examples, not the only possible embodiments and implementations.

Having described various aspects of the subject matter above, additional disclosure is provided below that may be consistent with the claims originally filed with this disclosure. In describing this additional subject matter, reference may be made to the previously described figures. Any of the following aspects may be combined, where compatible.

One general aspect includes a stethoscope sanitizing device for a stethoscope, a housing that at least partially surrounds a sanitation cavity. The stethoscope sanitizing device also may include a radiation emitter located within the housing, where the radiation emitter is configured to selectively emit a radiation into the sanitation cavity. The device also may include a support pane fixed to the housing and configured to receive and support a patient-facing side of a stethoscope.

Implementations may include one or more of the following features. The support pane may be transparent. The support pane may include an opening that exposes at least a portion of the patient-facing side of the stethoscope. The opening of the support pane may be circular, where the support pane forms a support ring configured to accommodate stethoscopes of different sizes. The reflective surface may form an inverted cone. The radiation emitter may be located at an apex of the cone. The chest piece receiving structure may be configured to secure a chest piece of the stethoscope in place when the chest piece receiving structure is located between a diaphragm of the stethoscope and a bell of the stethoscope. The housing may include a rim that extends axially beyond the support pane, where the rim at least partially surrounds the support pane to prevent radiation escape during device operation. The housing may include a chest piece receiving structure configured to secure the stethoscope via contact with a back surface of a chest piece of the stethoscope. The stethoscope sanitizing device may include at least one spring clip extending from the housing and configured to contact a chest piece of the stethoscope for securing the chest piece in place relative to the housing. The radiation emitter may include a uv-light emitter. The radiation emitter may be electrically connected to a controller located within the housing. The stethoscope may include a light collector that glows upon receiving radiation from the radiation emitter. The light collector may include an indicator tab that is exposed for view by a user.

Another general aspect includes a stethoscope sanitizing device for a stethoscope with a housing that at least partially surrounds a sanitation cavity. The stethoscope sanitizing device also may include a radiation emitter located within the housing, where the radiation emitter is configured to selectively emit a radiation into the sanitation cavity. The device also may include a support pane fixed to the housing and configured to receive and support a patient-facing side of a stethoscope, where the support pane may include a transparent material, and where the support pane may include a central opening configured to permit direct radiation application to the patient-facing side of the stethoscope from the emitter.

Implementations may include one or more of the following features. The opening of the support pane may be circular, where the support pane forms a support ring configured to accommodate stethoscopes of different sizes. The reflective surface may form an inverted cone. The radiation emitter may be located at an apex of the cone. The housing may include a rim that extends axially beyond the support pane, where the rim at least partially surrounds the support pane to prevent radiation escape during device operation.

Another general aspect includes a method, with may have one or more of the following steps: assembling a stethoscope sanitizing device for a stethoscope, the stethoscope sanitizing a housing that at least partially surrounds a sanitation cavity and a radiation emitter located adjacent to the reflective surface, where the radiation emitter is configured to selectively emit a radiation into the sanitation cavity. A support pane fixed to the housing may be included and configured to receive and support a patient-facing side of a stethoscope.

We claim:

1. A stethoscope sanitizing device for a stethoscope, comprising:
a housing that at least partially surrounds a sanitation cavity;
a radiation emitter located within the housing, wherein the radiation emitter is configured to selectively emit a radiation into the sanitation cavity; and
a support pane fixed to the housing and configured to receive and support a patient-facing side of a stethoscope.

2. The stethoscope sanitizing device of claim 1, wherein the support pane is transparent.

3. The stethoscope sanitizing device of claim 1, wherein the support pane includes an opening that exposes at least a portion of the patient-facing side of the stethoscope.

4. The stethoscope sanitizing device of claim 3, wherein the opening of the support pane is circular, and wherein the support pane forms a support ring configured to accommodate stethoscopes of different sizes.

5. The stethoscope sanitizing device of claim 1, further comprising a reflective surface at a bottom of the sanitation cavity, wherein the reflective surface forms an inverted cone.

6. The stethoscope sanitizing device of claim 5, wherein the radiation emitter is located at an apex of the cone.

7. The stethoscope sanitizing device of claim 1, wherein the housing includes a rim that extends axially beyond the support pane, wherein the rim at least partially surrounds the support pane to prevent radiation escape during device operation.

8. The stethoscope sanitizing device of claim 1, wherein the housing includes a chest piece receiving structure configured to secure the stethoscope via contact with a back surface of a chest piece of the stethoscope.

9. The stethoscope sanitizing device of claim 5, wherein the chest piece receiving structure is configured to secure a chest piece of the stethoscope in place when the chest piece receiving structure is located between a diaphragm of the stethoscope and a bell of the stethoscope.

10. The stethoscope sanitizing device of claim 1, further comprising at least one spring clip extending from the housing and configured to contact a chest piece of the stethoscope for securing the chest piece in place relative to the housing.

11. The stethoscope sanitizing device of claim 1, wherein the radiation emitter includes a UV-light emitter.

12. The stethoscope sanitizing device of claim 1, wherein the radiation emitter is electrically connected to a controller located within the housing.

13. The stethoscope of claim 1, further comprising a light collector that glows upon receiving radiation from the radiation emitter.

14. The stethoscope of claim 13, wherein the light collector includes an indicator tab that is exposed for view by a user.

15. A stethoscope sanitizing device for a stethoscope, comprising:
a housing that at least partially surrounds a sanitation cavity;
a radiation emitter located within the housing, wherein the radiation emitter is configured to selectively emit a radiation into the sanitation cavity; and
a support pane fixed to the housing and configured to receive and support a patient-facing side of a stethoscope,
wherein the support pane includes a transparent material, and
wherein the support pane includes a central opening configured to permit direct radiation application to the patient-facing side of the stethoscope from the emitter.

16. The stethoscope sanitizing device of claim 15, wherein the opening of the support pane is circular, and wherein the support pane forms a support ring configured to accommodate stethoscopes of different sizes.

17. The stethoscope sanitizing device of claim 15, further comprising a reflective surface at a bottom of the sanitation cavity, wherein the reflective surface forms an inverted cone.

18. The stethoscope sanitizing device of claim 17, wherein the radiation emitter is located at an apex of the cone.

19. The stethoscope sanitizing device of claim 15, wherein the housing includes a rim that extends axially beyond the support pane, wherein the rim at least partially surrounds the support pane to prevent radiation escape during device operation.

20. A method, comprising:
assembling a stethoscope sanitizing device for a stethoscope, the stethoscope sanitizing device comprising:
a housing that at least partially surrounds a sanitation cavity;
a radiation emitter located adjacent to the reflective surface, wherein the radiation emitter is configured to selectively emit a radiation into the sanitation cavity; and
a support pane fixed to the housing and configured to receive and support a patient-facing side of a stethoscope.
